(19) United States
(12) Patent Application Publication
(10) Pub. No.: US 2025/0372212 A1
(43) Pub. Date: Dec. 4, 2025

(54) MACHINE LEARNING-BASED METHOD AND SYSTEM FOR IDENTIFYING SUBPOPULATIONS IN CLINICAL STUDIES

(71) Applicant: PHASEV TRIALS LTD, Tel Aviv (IL)

(72) Inventors: Raviv PRYLUK, Ramat Gan (IL); Elad BERKMAN, Givataim (IL); Oshri MACHLUF, Ramat Gan (IL); Tzviel FROSTIG, Tel Aviv (IL); Gal SHOHAM LEVIN, Pardes Hanna-Karkur (IL)

(21) Appl. No.: 18/677,138

(22) Filed: May 29, 2024

(51) Int. Cl. G16H 10/20; G16H 50/70

(52) U.S. Cl. CPC G16H 10/20; G16H 50/70

(57) ABSTRACT

Disclosed is method and system for identifying treatment subpopulations within a patient population of a clinical study, by applying a causal ensemble model configured to output an ensemble Conditional Average Treatment Effect (eCATE).

### TECHNOLOGICAL FIELD

The present disclosure generally relates to a system and method for identifying subpopulations or biomarkers within a patient population of a clinical study.

### BACKGROUND

Clinical trials aim to estimate the safety and efficacy of a tested treatment, usually in comparison to a control (Standard or Care or Placebo). Efficacy is measured according to clinical outcomes which are commonly referred to as the trial's endpoints. The main measure of interest in terms of efficacy is the treatment effect, which is the expected difference in outcome under treatment vs outcome under control. However, for any individual patient, one can only observe one of these potential outcomes (either under treatment or under control), but never both—resulting in “the fundamental problem of causal inference”. Since for each patient only one potential outcome is observed, a natural way to infer about treatment effect is to use multiple patients—some given the treatment and some the control and compare their outcomes on average. If the assignment of treatment is independent of the potential outcome, then the difference between average outcome of the treated patients and the average outcome of the control patients equals the Average Treatment Effect (ATE). However, often, there is considerable variability in the treatment effect between different subgroups of patients due to underlying heterogeneity. This variability is one of the key reasons for failures when transitioning from Phase 2 to Phase 3 studies.

As a result, the identification of subgroups within the patient population is a paramount part of when analyzing clinical data. To this end localized estimates of a treatment effect—also referred to as Conditional Average Treatment Effect (CATE) is conducted. However, CATE analyses are often met with skepticism as their results are often not replicated in future studies. Moreover, computing a CATE can be very challenging in particular in exploratory clinical phases, in which significant clinical information is accumulated for the first time, and critical decisions about the target population of a treatment need be made, often based on relatively small sample sizes (especially in Phase II exploratory trials).

There therefore remains a need for an improved computation of CATE that can reliably identify subpopulations in clinical studies.

### SUMMARY

According to some embodiments, there is provided a method for identifying one or more treatment subpopulations within a patient population of a clinical study, by applying on a dataset obtained from the clinical study a causal ensemble model, the ensemble model integrating at least two different causal predictive models to obtain an improved CATE, also referred to herein as “eCATE”.

Advantageously, the herein disclosed approach enables identifying subgroups of “wide data”, i.e. data including many covariates relative to the sample size.

The approach can advantageously detect even subtle signals thereby capturing variability at a high resolution. This in turn facilitates discovering personalized treatments which take individual patient differences into account.

Furthermore, the herein disclosed method and system enables not only to predict treatment outcomes, but also aids in understanding the mechanisms through which the treatments work, thus further paving the way for more personalized and effective therapies.

Causal predictive models have been suggested for CATE calculation. However, no single model can serve as a reliable predictor across scenarios, and it is typically impossible to know which model will be the most efficient for a particular dataset. Advantageously,

Moreover by integrating at least two different causal predictive models into a universal ensemble algorithm, a methodological synergy between the models is unexpectedly achieved.

According to some embodiments, there is provided a method for identifying one or more treatment subpopulations within a patient population of a clinical study, the method including:
a. obtaining a dataset comprising a measured treatment response for each individual in at least one drug treated patient group and a measured treatment response for each individual in a control treated patient group, wherein each individual in the drug treated group and the control group is characterized by a plurality of features, thereby forming a multidimensional feature space;
b. applying at least two different causal predictive models on the dataset, each causal predictive model configured to output a Conditional Average Treatment Effect (CATE) for each point in the multidimensional feature space;
c. integrating the at least two different causal predictive models into a causal ensemble model configured to output an ensemble Conditional Average Treatment Effect (eCATE) for each point in the multidimensional feature array,
d. identifying one or more subpopulations within the patient population, based on the eCATE and their associated features.

According to some embodiments, at least one of the at least two causal predictive models is a meta-learner. According to some embodiments, the applying of the at least two causal predictive models includes computing a counterfactual control treatment response for each individual in the at least one drug treated patient group and a counterfactual treatment response for each individual in the control drug treated patient group. According to some embodiments, computing the counterfactual treatment response includes, for each individual in the at least one drug treated group, inputting his/her features into a model trained on the control treated group, and for each individual in the control treated group, inputting his/her features into a model trained on the drug treated group. According to some embodiments, the method of further includes computing a hypothetical individual treatment effect (ITE) for each individual in the dataset, based on a difference between the measured treatment response and the counterfactual treatment response of each individual in the training set.

According to some embodiments, at least one of the at least two causal predictive models is a causal-forest or a causal tree learner. According to some embodiments, at least one of the at least two causal predictive models is derived using a direct estimation method. According to some embodiments, the causal ensemble model configured output a predicted treatment response for each point in the multi-dimensional space.

According to some embodiments, the method further includes computing a confidence interval for each of the at least two causal predictive models.

According to some embodiments, the eCATE is a simple average eCATE computed by averaging the CATE of each the at least two different causal predictive models. According to some embodiments, the eCATE is a weighted average eCATE computed by averaging the CATE of each the at least two different causal predictive models while weighing according to the computed confidence interval. According to some embodiments, the eCATE is a consensus-based (CBA) eCATE. According to some embodiments, computing the CBA eCATE includes averaging the CATE of the predictive models out of the at least two causal predictive models having a computed confidence interval within a predetermined threshold value only. According to some embodiments, computing the CBA eCATE includes averaging the CATE of the predictive models out of the at least two causal predictive models identifying a same group of features as influencing the CATE only.

According to some embodiments, the at least two causal predictive models are selected from generalized linear model (GLM), Accurate GLM, Causal Forest, Regression Trees, Boosted Regression Trees, Random Forest, Bayesian Additive Regression Trees (BART), Neural Networks deep learning methods (TAR-Net), non-parametric methods such as Gaussian Process regression, Causal Graphical Models or any combination thereof. Each possibility is a separate embodiment.

According to some embodiments, the at least two causal predictive models are selected from Accurate GLM, Causal Forest, Random Forest, Bayesian Additive Regression Trees (BART), or any combination thereof. Each possibility is a separate embodiment.

According to some embodiments, the at least two causal predictive models comprise at least three, at least four or at least five causal predictive models. Each possibility is a separate embodiment.

According to some embodiments, the dataset is a clinical trial dataset, an observational dataset, a real-world dataset or any combination thereof. Each possibility is a separate embodiment.

According to some embodiments, the method further includes outputting, e.g. by displaying on a display, the identified subgroups and their identifying features.

According to some embodiments, there is provided a system including a memory and a processor coupled to the memory programmed with executable instructions, configuring the processor to:
a. obtain a dataset comprising a measured treatment response for each individual in at least one drug treated patient group and a measured treatment response for each individual in a control treated patient group, wherein each individual in the drug treated group and the control group is characterized by a plurality of features, thereby forming a multidimensional feature space;
b. apply at least two different causal predictive models on the dataset, each causal predictive model configured to output a Conditional Average Treatment Effect (CATE) for each point in the multidimensional feature space;
c. integrate the at least two different causal predictive models into a causal ensemble model configured to output an ensemble Conditional Average Treatment Effect (eCATE) for each point in the multidimensional feature array,
d. identifying one or more subpopulations within the patient population, based on the eCATE and their associated features.

Advantageously, the herein disclosed system provides improved processing capabilities to the processor thus allowing it to reliably and robustly identify patient subgroups with patient populations of a clinical study.

According to some embodiments, the processor is configured to output, e.g. on a display, the identified patient sub-groups and their common features.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

Some embodiments of the disclosure are described herein with reference to the accompanying figures. The description, together with the figures, makes apparent to a person having ordinary skill in the art how some embodiments may be practiced. The figures are for the purpose of illustrative description and no attempt is made to show structural details of an embodiment in more detail than is necessary for a fundamental understanding of the disclosure. For the sake of clarity, some objects depicted in the figures are not drawn to scale. Moreover, two different objects in the same figure may be drawn to different scales. In particular, the scale of some objects may be greatly exaggerated as compared to other objects in the same figure.

In block diagrams and flowcharts, certain steps may be conducted in the indicated order only, while others may be conducted before a previous step, after a subsequent step or simultaneously with another step. Such changes to the orders of the step will be evident for the skilled artisan.

FIG. 1 is an exemplary flowchart of the herein disclosed method for identifying one or more treatment subpopulations within a patient population of a clinical study;

FIG. 2 is a bar graph showing the excess scaled (Root Mean Square Error) RMSE (vs. best) of various CATE estimators as compared to the herein disclosed CATE ensemble model.

### DETAILED DESCRIPTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, disclosed is a method for identifying one or more treatment subpopulations within a patient population of a clinical study.

As used herein the term “clinical study” refers to research studies that test how well medical approaches work in people.

According to some embodiments, the clinical study may be a clinical trial. As used herein, the term “clinical trial” refers to prospective biomedical (or behavioral) research studies on human participants designed to answer specific questions about new treatments. They generate data on dosage, safety and efficacy and typically include four phases. According to some embodiments, the clinical trial may be an exploratory phase II clinical trial.

According to some embodiments, the clinical study may be an observational study. As used herein, the term “observational study” refers to a study in which events, behaviors, or phenomena are recorded as they naturally occur without interference or manipulation.

According to some embodiments, the clinical study may be a real-world study. As used herein the term, “real world study” refers to the collection of Real-world data (RWD) i.e. data relating to patient health status routinely collected from a variety of sources. RWD can be generated from: Electronic health records, medical claims, billing data, insurance data, data from product and disease registries, patient-generated data, data gathered from mobile devices etc.

According to some embodiments, the method includes obtaining a dataset including a measured treatment response for each individual in at least one treated patient group of the clinical study and a measured treatment response for each individual in a control patient group of the clinical study. According to some embodiments, each individual in the drug treated group and in the control group is characterized by a plurality of features, which form a multidimensional feature space.

According to some embodiments, the term “control patient group” may refer to a group of patients receiving no treatment. According to some embodiments, the term “control patient group” may refer to a group of patients receiving a placebo treatment. According to some embodiments, the term “control patient group” may refer to a group of patients receiving standard care (SoC). According to some embodiments, the term “control patient group” may refer to a hypothetical group of patients derived from real world data (RWD), e.g. using matching algorithms. For example, in single arm studies (e.g. single arm oncology studies), a randomized clinical trial may be achieved by deriving matching SoC data from RWD or from other clinical trials.

As used herein the term “measured treatment response” refers to the actual treatment response measured for an individual. According to some embodiments, the measured treatment response is a function of a vector of the patient's features/covariates and the treatment allocation (e.g. treatment I, treatment II or control). According to some embodiments, the measured treatment response can be mirrored by a “hypothetical treatment response”, also referred to herein as a “counterfactual treatment response”. For example, if a patient is allocated to a treatment group (for which he/she has a measured treatment response) a hypothetical response may be computed for the same individual, as further elaborated herein.

According to some embodiments, the term “at least one”, with respect to treatment groups (also referred to as “arms”) of a clinical study, may refer to a clinical study including a single treatment group, two treatment groups (e.g. first medicament and second medicament, first dose and second dose etc.), three treatment groups, four treatment groups, five treatment groups or more. Each possibility is a separate embodiment. According to some embodiments, a clinical study including more than one treatment arm may include a single control group (e.g. standard care or placebo). According to some embodiments, a clinical study including more than one treatment arm may include a control group for each arm.

According to some embodiments, the term “plurality” with respect to the features refers to at least 3 features, at least 5 features, at least 10 features, at least 15 features or more. Each possibility is a separate embodiment. According to some embodiments, the plurality of features may include three or more of: age, sex, weight, height, ethnicity, medical background, marital status, geographic location, socio-economic status, heart rate at rest, saturation, diet, number of children, number of pregnancies, number of unforced abortions, genomic signatures such as PD1 levels, metabolic signatures, previous medications and the like. Each possibility and combination of possibilities is a separate embodiment.

As used herein, the term “multidimensional feature space” refers to the space generated by the multiple combination of features (also referred to as “covariate”) characterizing each individual in the clinical study (e.g. female, age 50, weight 70 kg, married with 5 children etc.) as well as hypothetical combinations (i.e., combinations of features that are possible but not represented by any of the individuals in the clinical study.

According to some embodiments, the method includes applying at least two different causal predictive models on the dataset. According to some embodiments, each causal predictive model configured to output a predicted Conditional Average Treatment Effect (CATE) for each point in the multidimensional feature space (real and/or hypothetic).

As used herein, the terms “Average Treatment Effect” and “ATE” refer to the difference between an average outcome of treated patients and an average outcome of control patients. Assuming that the assignment of treatment is independent of the potential outcomes, ATE can be expressed as:

$$E [ y ( a = 1 ) - y ( a = 0 ) ] ; \\ if : a \{ y ( a = 0 ) , y ( a = 1 ) \} = E [ y \vert a = 1 ] - E [ y \vert a = 0 ]$$

Where a is the treatment assignment (0 for control, 1 for treatment), and y (a=i) is the outcome under treatment assignment i.

As used herein, the terms “Conditional Average Treatment Effect” and “CATE” refer to a difference between the expected outcomes of the two treatments conditioned on covariates, i.e. the average effect of a treatment on a sub-group, wherein the validity of the estimate is conditional on being part of this subgroup. CATE is distinct from ATE, which is the average treatment effect on an entire study population.

CATE can be expressed as:

$$τ ( Xi ) = E [ y ( a = 1 ) - y ( a = 0 ) \vert X = Xi ]$$

based on the patients features; by definition τ(X)=E [τi|X]

As used herein, the term “causal predictive models” and “predictive causal models” may be used interchangeably and refer to machine learning (ML) models that relate independent variables (i.e. variables which can be manipulated) to dependent variables (variables that can be measured), generating predictions for the values of dependent variables given a set of values for the independent variables. According to some embodiments, the at least two predictive models may be causal forests and/or meta learners.

Causal forest (CF) is an adaptation of random forests, in which the base trees composing the forest are causal trees, aimed at estimating local differences between average potential outcomes. According to some embodiments, the CF is a CF with double/debiased machine learning. Double/debiased machine learning (DML) is a method developed to use regularized regression techniques for variable selection in a high-dimensional causal inference setting. It seeks variables that are highly correlated with both treatment and outcome, thereby reducing small approximation errors that arise when selecting among a large set of covariates.

Meta-Learners are an estimation framework that enables using any ML model as a “base learner” for learning various nuisance functions and composing an estimator for CATE using a transformation of the learned functions. There are several common meta-learner structures, including, but not limited to:
S learner: A single (hence “S”) model is trained to regress the outcomes on the features and the treatment assignment (the treatment is treated as an additional binary variable attached to X).

$$y \hat{} ( x , α ) = E \hat{} [ y ( X , a ) \vert X = x , a = α ]$$

CATE is the estimated by contrasting this model's predictions for both potential outcomes:

$$τ \hat{} ( X ) = y \hat{} ( X , 1 ) - y \hat{} ( X , 0 )$$

T learner: This approach uses base-learners to estimate the conditional expectations of the two (hence “T”) potential outcomes—{(Xi, yi); ai=0} that are used to train \hat{ }μ0(X), an estimate for E[y|a=0] and {(Xi, yi); ai=1} to train \hat{ }μ1(X). Finally, an estimate for CATE is obtained by subtracting them:

$$τ \hat{} ( X ) = μ \hat{} 1 ( X ) - μ \hat{} 0 ( X )$$

X learner: This approach builds on the foundations of the T Learner and starts similarly by estimating μ 0(X) and \hat{ }μ1(X). it then uses these estimates to impute the missing potential outcomes and generate “pseudo individual effects”:

$$if ai = 0 : D 0 i := μ \hat{} 1 ( Xi ) - yi \\ if ai = 1 : D 1 i := yi - μ \hat{} 0 ( Xi )$$

Next, {D 0 i} and {D 1 i} are used to train two separate estimates for CATE—\hat{ }τ1(X) using {D 1 i} and \hat{ }τ0(X) using {D 0 i}. Finally, a weighted average of the two estimates is used to estimate CATE.
DR learner: This approach constructs a doubly-robust pseudo-outcome for CATE using a sub-sample of the training data, and uses the rest of the train-set to regress this pseudo outcome on X. First, using the first subset S1 to train \hat{ }π(X), μ\hat{ }0(X), μ\hat{ }1(X)—estimates for π(X), E[y|a=0, X], E[y|a=1, X], respectively. Then, the following pseudo-outcome is constructed:

$$φ \hat{} ( X , y , a ) = a - π \hat{} ( X ) π \hat{} ( X ) \cdot ( 1 - π \hat{} ( X ) ) ( y - μ \hat{} a ( X ) ) + μ \hat{} 1 ( X ) - μ \hat{} 0 ( X )$$

Finally, the rest of the train-sample, S2=S\S1, is used to regress:

$$τ \hat{} dr ( x ) = E \hat{} [ φ \hat{} ( X , y , a ) \vert X = x ]$$

Each of the aforementioned types of causal predictor model can use any ML model for any of the nuisance functions it estimates. The choice of base estimators should account for the assumed complexity of the underlying data-generating process, the size of available training data etc. Clinical trials often test complex mechanisms, which favor more flexible ML models. However, the typical sample sizes in this setting are usually quite limited, favoring simpler more tightly regulated models.

According to some embodiments, the at least two different causal predictive models (MLs) may be selected from:
GLMs: Regularized (Lasso and Elastic-Net) linear regression is used to estimate outcome and pseudo-outcomes, and logistic regression to estimate the propensity.
Accurate GLM (AGLM): This method utilizes Lasso regression to fit a piece-wise constant function, by first encoding each variable into nested bins.
Boosted Regression Trees: A sum of trees, where each tree is fitted on the residuals of the previous one.
Random Forests (RF): An average of trees, constructed with stochastic sampling of features and sample to induce variability in the trees, which acts as a form of regularization.
Bayesian Additive Regression Trees (BART): inspired by ensemble methods, with boosting in particular, BART also trains a sum-of-trees model, with the addition of a regularization prior which controls the parameters of that model.
Regression trees: decision tree algorithms where the target variable can take continuous values.
Non-parametric methods such as gaussian process regression, or spline regression.

According to some embodiments, the at least two causal predictive models are integrated into a causal ensemble model configured to output an “ensemble Conditional Average Treatment Effect” (also referred to herein as “eCATE”).

According to some embodiments, the eCATE is computed by averaging the CATE of each the at least two different causal predictive models, also referred to herein as “a simple average eCATE”.

According to some embodiments, the eCATE is computed by averaging the CATE of each the at least two different causal predictive models while weighing according to a computed confidence interval of each model, such that models that output a CATE with a higher confidence interval get at larger weight—in which case the eCATE is also referred to herein as “a weighted average eCATE”.

According to some embodiments, the eCATE is a consensus-based (CBA) eCATE. According to some embodiments, the CBA-eCATE may be computed by only averaging the CATE of the predictive models having a confidence interval within a predetermined threshold value, while ignoring outlier models. According to some embodiments, the CBA-eCATE may be computed by only averaging the CATE of the predictive models that identify the same features as influencing the CATE (e.g. averaging only models that identify gender, age and weight is influencers of CATE while ignoring models that identify other features (different from those of other models) e.g. height.

According to some embodiments, the method further includes identifying one or more drug subpopulations within the patient population, based on the eCATE and their associated features and optionally planning future clinical studies, based on the stratification into sub-populations.

Although some embodiments of the invention are not limited in this regard, discussions utilizing terms such as, for example, “processing,” “computing.” “calculating,” “determining,” “establishing”. “analyzing”, “checking”, “detecting”, “identifying”, “characterizing”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term set when used herein may include one or more items. Unless explicitly stated, the method embodiments described herein are not constrained to a particular order or sequence. Additionally, some of the described method embodiments or elements thereof can occur or be performed simultaneously, at the same point in time, or concurrently.

As used herein, the terms “approximately”, “essentially” and “about” in reference to a number are generally taken to include numbers that fall within a range of 5%, 2.5% or in the range of 1% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

As used herein, the singular forms “a,” “an” and “the” include plural referents unless the context clearly dictates otherwise.

As used herein, “optional” or “optionally” means that the subsequently described event or circumstance does or does not occur, and that the description includes instances where said event or circumstance occurs and instances where it does not.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination or as suitable in any other described embodiment of the disclosure. No feature described in the context of an embodiment is to be considered an essential feature of that embodiment, unless explicitly specified as such.

Although stages of methods, according to some embodiments, may be described in a specific sequence, the methods of the disclosure may include some or all of the described stages carried out in a different order. In particular, it is to be understood that the order of stages and sub-stages of any of the described methods may be reordered unless the context clearly dictates otherwise, for example, when a latter stage requires as input an output of a former stage or when a latter stage requires a product of a former stage. A method of the disclosure may include a few of the stages described or all of the stages described. No particular stage in a disclosed method is to be considered an essential stage of that method, unless explicitly specified as such.

Although the disclosure is described in conjunction with specific embodiments thereof, it is evident that numerous alternatives, modifications, and variations that are apparent to those skilled in the art may exist. Accordingly, the disclosure embraces all such alternatives, modifications, and variations that fall within the scope of the appended claims. It is to be understood that the disclosure is not necessarily limited in its application to the details of construction and the arrangement of the components and/or methods set forth herein. Other embodiments may be practiced, and an embodiment may be carried out in various ways.

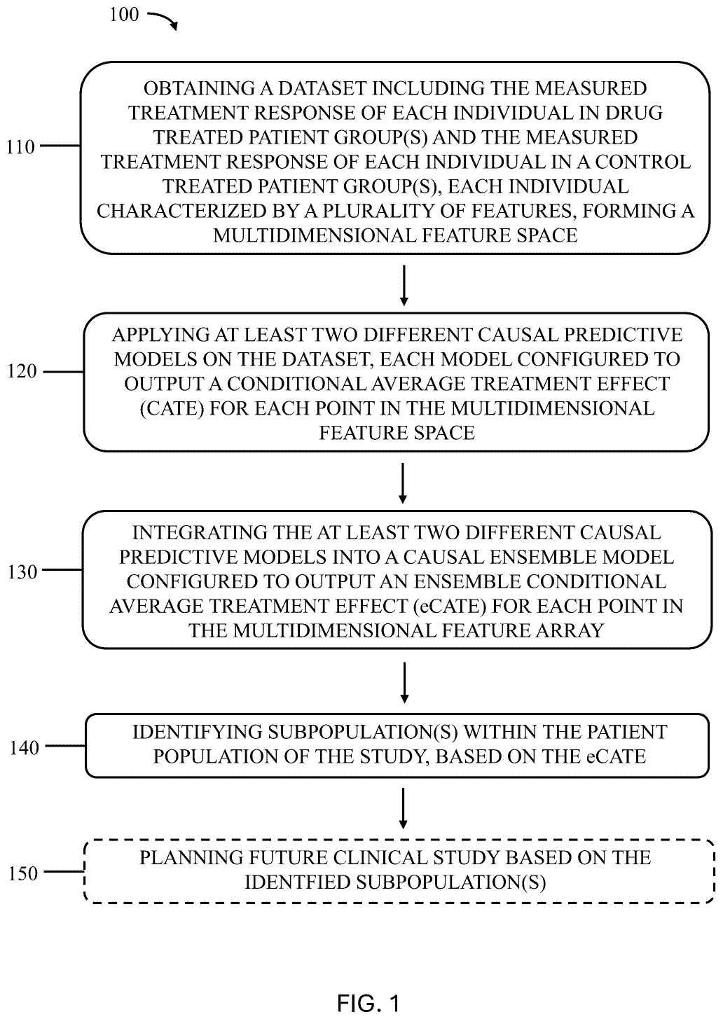

Reference is now made to FIG. 1, which is an exemplary flowchart of the herein disclosed method 100 for identifying treatment subpopulation(s) within a patient population of a clinical study.

In step 110, a clinical dataset is obtained, the dataset including the measured treatment response for each individual in one or more drug treated patient groups and the measured treatment response for each individual in one or more control treated patient group, wherein each individual in each of the groups is characterized by a plurality of features, forming a multidimensional feature space.

In step 120, at least two different causal predictive (ML) models are each applied on the dataset, each model configured to output a Conditional Average Treatment Effect (CATE) for each point in the multidimensional feature space, whether the point reflects an actual or hypothetical patient. As a non-limiting example, the at least two different causal predictive (ML) models may be XLearner (BART), Xlearner (RF), CF and AGLM.

In step 130, the at least two different causal predictive models are integrated into a causal ensemble model configured to output an ensemble Conditional Average Treatment Effect (eCATE) for each point in the multidimensional feature array. According to some embodiments, the integration comprises a simple averaging of the CATE of each the at least two different causal predictive models. According to some embodiments, the integration comprises computing a weighted average of the CATE of each the at least two different causal predictive models, wherein the weighting is carried out for example based on the confidence interval of each model and/or the predictive strength of each model. According to some embodiments, the integration comprises consensus-based averaging, e.g. averaging the CATE only of models that having a confidence interval within a predetermined threshold value and/or averaging the CATE only of models that identify the same dominant covariates.

In step 140, one or more subpopulations are identified within the patient population, based on the eCATE and their associated features. According to some embodiments, the subpopulation may be a subpopulation or subpopulations that are sensitive to a specific treatment arm. According to some embodiments, the subpopulation may be a subpopulation or subpopulations that do not respond to (any) treatment and as such should be excluded.

Optionally, method 100 may include an additional step 150 of planning a future clinical study (e.g. a phase III clinical trial) based on the identified subpopulation or subpopulations. According to some embodiments, the planning may include choosing an optimal number of individuals belonging to a specific subpopulation. According to some embodiments, the planning may include excluding subpopulations from the planned study etc.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1—Comparative Study of CATE Calculation

The focus of this study is a comprehensive analysis of the performance of different CATE estimation models in different scenarios. Since CATE is not directly observable in real patients, simulated data is required in order to test the performance in a valid and reliable manner. A simulation study on synthetic data, and a complementary analysis of real-world clinical trials data, were thus conducted.

### 1.1 Scenario Simulation

In order to evaluate the robustness of the models, two data generating mechanisms, based on a linear model or a linear model with addition of nonlinear transformations and/or second order interactions between features were tested, representing three type of scenarios, namely: Linear scenarios, slightly nonlinear scenarios and highly nonlinear scenarios.

In addition, a second data-generating mechanism utilizing a mechanistic disease model of a main pathway or mechanism of action for a selected disease. Here, a simplified mechanistic model for the PD-L1 pathway in urothelial cancer was utilized. This model describes the connections between a few key factors related to the progression of urothelial tumors and specifically to their treatment using PD-L1 inhibitors.

### 1.2 Estimators

Two types of estimators were applied, namely causal forests and meta-learners and base-learners spanning a wide range in complexity were compared:
Boosted Regression Trees: A sum of trees, where each tree is fitted on the residuals of the previous one.
Accurate GLM (AGLM): This method utilizes Lasso regression to fit a piece-wise constant function, by first encoding each variable into nested bins.
Random Forests (RF): An average of trees, constructed with stochastic sampling of features and sample to induce variability in the trees, which acts as a form of regularization.
Bayesian Additive Regression Trees (BART): with boosting in particular, BART also trains a sum-of-trees model, with the addition of a regularization prior which controls the parameters of that model.
Non-parametric methods such as gaussian process regression, or spline regression.
Neural Networks algorithms, such as TAR-net

These estimators were compared to interaction testing, which is the traditional approach used to analyze heterogeneity and identify subgroups in clinical data.

The abovementioned estimators, as well as the interaction testing were further compared to the herein disclosed ensemble model, also referred to as a CATE ensemble model, integrating the causal forest, AGLM and BART. Two types of integrations were tested namely, integration based on a simple mean of the predictions (results not shown), and a Consensus Based Averaging (CBA) approach, which identifies a cluster of “most concordant” models, and averages only them.

### 1.3 Data Training

In contrast to outcome prediction, where it is possible to compare the predicted outcome for an observation to its measured outcome, in the case of CATE estimation, this is impossible due to the fact that the treatment effect cannot be directly measured for any patient, since only one potential outcome can be observed for each patient. This was solved inter alia by using synthetic datasets to train and test models, which allows us to compare CATE estimation to the real (known) CATE for any individual of interest.

In short, the models were trained on a training data set, and its performance evaluated on a held-out test set generated using the same synthetic data generating process (DGP) by Root Mean Squared Error RMSE. RMSE measures the distance between predictions and actual CATE. The RMSE was scaled by the standard deviation of the CATE.

### 1.4 Results

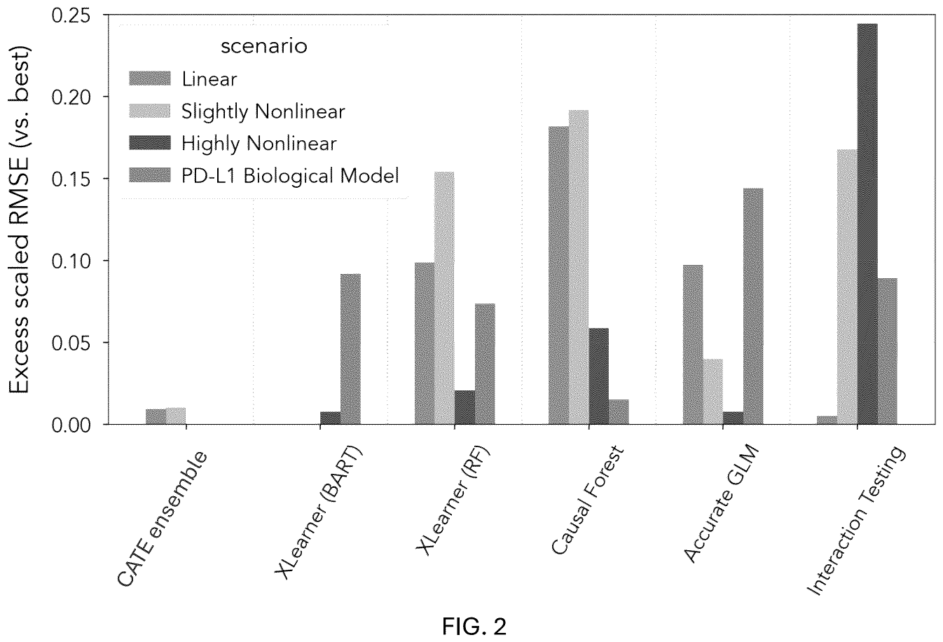

As seen from FIG. 2, none of the single estimators had good performance (compared to others) in all scenarios, and every single estimator failed significantly in at least one of the scenario types. This, as opposed to the CATE ensemble model, which performed remarkably well in all scenarios, in particular in highly non-linear scenarios and biological models.

These results clearly demonstrate the superiority of the herein disclosed model in deriving reliable and robust CATEs from clinical studies and thus for providing a reliable prediction of treatment efficacy. Hence, the herein disclosed CATE ensemble model leverages the strengths of multiple causal inference techniques and ensures superior performance and capability of handling complex, heterogeneous datasets effectively.

The ability of the herein disclosed ensemble algorithm to account for patient heterogeneity significantly improves the analysis of clinical trials and thus bolsters the chances of drug approval. Moreover, by providing a more accurate assessment of how different patient groups will respond to treatments, the herein disclosed ensemble approach enables personalized medicine strategies that are more effective and efficient. This not only optimizes resource use but also improves patient care by tailoring treatments to individual needs, thus advancing the field of personalized medicine.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. A method for identifying one or more treatment subpopulations within a patient population of a clinical study, the method comprising:
a. obtaining a dataset comprising a measured treatment response for each individual in at least one drug treated patient group and a measured treatment response for each individual in a control treated patient group, wherein each individual belongs either to the drug treated group or to the control group, and wherein each individual in the drug treated group and the control group is characterized by a plurality of features, thereby forming a multidimensional feature space;
b. applying at least two different causal predictive machine learning (ML) models on the dataset, each causal predictive ML model configured to output a Conditional Average Treatment Effect (CATE) for each point in the multidimensional feature space;
c. converting the at least two different causal predictive models into a single causal ensemble model by averaging an individual CATE of each of the at least two different causal predictive models while weighing the individual CATE according to their respective computed confidence interval,
d. training the causal ensemble model on a dataset comprising a counterfactual treatment response for each individual in the at least one drug treated patient group and the control treated patient group, thereby generating a trained causal ensemble model,
e. utilizing the trained causal ensemble model to:
i. compute an ensemble Conditional Average Treatment Effect (eCATE) for each point in the multidimensional feature space,
ii. identify one or more subpopulations within the patient population, based on the eCATE and their associated features; and
iii. automatically produce a clinical study plan comprising sample size and stratification factors, based on the identified subpopulation or subpopulations; and
f. conducting a clinical study using the produced clinical study plan.

2. The method of claim 1, wherein at least one of the at least two causal predictive models is a meta-learner.

3. (canceled)

4. The method of claim 1, further comprising computing the counterfactual treatment response comprises for each individual in the at least one drug treated group, by inputting his/her features into a causal ensemble model trained on the control treated group, and for each individual in the control treated group, by inputting his/her features into a causal ensemble model trained on the drug treated group.

5. The method of claim 1, further comprising computing a hypothetical individual treatment effect (ITE) for each individual in the dataset, based on a difference between the measured treatment response and the counterfactual treatment response of each individual in the training set.

6. The method of claim 1, wherein at least one of the at least two causal predictive models is a causal-forest or a causal tree learner.

7. The method of claim 1, wherein at least one of the at least two causal predictive models is derived using a direct estimation method.

8. The method of claim 1, wherein the causal ensemble model configured output a predicted treatment response for each point in the multi-dimensional space.

9. The method of claim 1, further comprising computing the confidence interval for each of the at least two causal predictive models.

10. (canceled)

11. (canceled)

12. The method of claim 1, wherein the at least two different causal predictive models into the single causal ensemble model further comprises applying a consensus-based (CBA) eCATE.

13. The method of claim 12, wherein computing the CBA eCATE comprises averaging the CATE of the predictive models out of the at least two causal predictive models having a computed confidence interval within a predetermined threshold value only.

14. The method of claim 12, wherein computing the CBA eCATE comprises averaging the CATE of the predictive models out of the at least two causal predictive models identifying a same group of features as influencing the CATE only.

15. The method of claim 1, wherein the at least two causal predictive models are selected from generalized linear model (GLM), Accurate GLM, Causal Forest, Regression Trees, Boosted Regression Trees, Random Forest, Bayesian Additive Regression Trees (BART), Neural Networks deep learning methods, non-parametric methods such as Gaussian Process, Causal Graphical Models.

16. The method of claim 1, wherein the at least two causal predictive models comprise at least three causal predictive models.

17. The method of claim 1, wherein the dataset is a clinical trial dataset, an observational dataset, a real-world dataset or any combination thereof.

18. The method of claim 1, wherein the plurality of features comprises at least 10 features.
